(19) United States
(12) Patent Application Publication
(10) Pub. No.: US 2005/0094348 A1
(43) Pub. Date: May 5, 2005

(54) Human body earthing apparatus

(76) Inventors: Masato Hattori, Sendai-shi (JP); Nobuyuki Ohba, Sendai-shi (JP)

Correspondence Address: SNIDER & ASSOCIATES, P. O. BOX 27613, WASHINGTON, DC, 20038-7613, US

(21) Appl. No.: 10/972,364

(22) Filed: Oct. 26, 2004

(30) Foreign Application Priority Data
Oct. 29, 2003 (JP) 2003-369670

(51) Int. Cl. H02B 1/00; A61N 2/00

(52) U.S. Cl. 361/220

(57) ABSTRACT

To provide a human body earthing apparatus capable of stably obtaining a high effect without using exclusive technique or knowledge.

One end of an earth line 11 is constituted of two branch lines 15 and one end of each branch line 15 has a human body earthing apparatus 17 and the other end of it has a ground connecting portion 18. A high-frequency-current limiter 13 is connected to the earth line 11 in series and has a configuration capable of selectively switching the frequency band of a high-frequency current which can be cut off. A current-backflow prevention unit 12 is connected to each branch line 15 in series. A current-direction controller 14 is connected to the other end of the earth line 11 in series. The current-direction controller 14 has a first diode 23 for supplying current from one end of to the other end of the earth line 11, a second diode 24 for supplying current from the other end of to one end of the earth line 11, a switching earth line 25 for supplying current in both directions, and a switch 26 capable of selectively switching the first diode 23, second diode 24, and switching earth line 25.

### RELATED APPLICATIONS

This application claims the priority of Japanese Patent Application No. 2003-369670, filed on Oct. 29, 2003, which is incorporated herein by reference.

### BACKGROUND OF THE INVENTION

1. Field of the Invention

The present invention relates to a human body earthing apparatus for eliminating excess or deficiency of ions or electrons from a human body.

2. Description of the Related Art

It is known that electrons are produced when atoms of Na, Ca, and K are ionized to serve as electrical energies of various activities in a living body. A cardiogram, brain wave, and electromyogram measure a weak potential difference between these electrical energies. Moreover, when a cell is damaged and a pain is felt, it is known that electrons are collected on the place and this is referred to as injury current. Furthermore, in the case of a chronic cloddish sense, local paralysis, and uncomfortable sense, it is known that electrons run short, activities of cells become dull, and blood flow is deteriorated.

An ion pumping therapy and a diode therapy have been used so far as methods for performing medical treatment by eliminating excess or deficiency of ions and electrons in the living body. In the case of the ion pumping therapy, a needle is applied to an pain portion and a portion separate from the pain portion and both needles are connected by a cord having a built-in diode (refer to Japanese Utility Model Laid-Open No. 50-64387 and Japanese Utility Model Laid-Open No. 50-64388) to neutralize excessive cations of the pain portion. In the case of the diode therapy, a diode is applied to an acupuncture point to obtain the same effect.

Moreover, as a conventional human-body earthing apparatus, there is an apparatus having an earth line and an electrode connected to the earth line and contacted with a human body to eliminate static electricity accumulated in the human body (refer to Japanese Patent Laid-Open No. 7-275371).

However, because the ion pumping therapy performs medical treatment by connecting applied needles, there is a problem that exclusive knowledge and technique of acupuncture and moxibustion are necessary. The diode therapy also has a problem that exclusive knowledge is necessary because of applying a diode to an acupuncture point. Moreover, the conventional apparatus disclosed in Japanese Patent Laid-Open No. 7-275371 is effective for elimination of static electricity but it has a problem that the effect for eliminating excess or deficiency of ions and electrons in a human body is not sufficient because it fluctuates.

The present invention is made to solve the above problems and its object is to provide a human body earthing apparatus capable of stably obtaining a high effect without using exclusive technique or knowledge.

### SUMMARY OF THE INVENTION

To achieve the above object, a human body earthing apparatus of the present invention includes an earth line whose one end has a human body connecting portion and whose other end has a ground connecting portion and a high-frequency-current limiter connected to the earth line in series.

A human body earthing apparatus of the present invention is used by bringing a human body connecting portion into contact with a human body and earthing an earth connecting portion. In this case, because electrons are moved from the human body to the ground or from the ground to the human body depending on the state of excess or deficiency of ions and electrons in the human body, it is possible to eliminate excess or deficiency of ions and electrons in the human body. Moreover, when the human body is electrified by static electricity, because electrons are moved from the human body to the ground and from the ground to the human body, it is possible to remove static electricity.

As a result of experiments with a human body earthing apparatus of the present invention, the present inventor et al. found that pain and paralysis due to excess or deficiency of ions and electrons in a human body could be eliminated by eliminating excess or deficiency of ions and electrons in human body. Moreover, as a result of experiments, we found that it was possible to reduce or eliminate pain by moving electrons from a human body to the ground when electrons are excessive by the pain and when electrons were insufficient due to chronic cloddish sense or paralysis and it is possible to reduce or eliminate the cloddish sense or paralysis by moving electrons from the ground to the human body.

Furthermore, as a result of using a conventional human-body earthing apparatus, the present inventor et al. found that the effect for eliminating excess or deficiency of ions and electrons in a human body fluctuated and was not sufficient. Moreover, because the fluctuation is changed depending on an environment of using the apparatus, the present inventor et al. estimated that it was a cause that a high-frequency current flew through an earth line from a television, radio set, cellphone, personal computer, or other radio unit to interrupt the flow of electrons or regurgitated electrons. Therefore, the present inventor et al. directly connected a high-frequency-current limiter to an earth line in order to cut off the high-frequency current and thereby, the effect of stably eliminating excess or deficiency of ions and electrons in a human body was obtained compared to the case of not connecting the high-frequency-current limiter. Thus, the present invention was completed.

Thus, in the case of a human body earthing apparatus of the present invention, a high-frequency-current limiter can cut off a high-frequency current from a radio unit flowing through an earth line. Therefore, it is possible to prevent the influence of the high-frequency current and stably obtain a high effect for eliminating excess or deficiency of ions and electrons in a human body. It is preferable that the high-frequency-current limiter is constituted of a line filter using ferrite beads for a coil. The line filter selectively separates signals and is able to cut off a high-frequency current like a low-pass filter and pass a DC current. Moreover, when using ferrite beads, it is possible to easily make a small and strong line filter.

In the case of a human body earthing apparatus of the present invention, it is preferable to bring a human body connecting portion into contact with an arch at the back of foot or a pain or paralysis portion. It is preferable that the human body connecting portion is constituted of a conductive plate such as a metallic plate. A human body earthing apparatus of the present invention can be easily used because exclusive technique or knowledge such as the case of acupuncture or moxibustion is unnecessary.

In the case of a human body earthing apparatus of the present invention, it is preferable that one end of the earth line is constituted of two branch lines and the end of each branch line has a human body connecting portion, and a current-backflow prevention unit connected to each branch line in series is included. In this case, because two human-body connecting portions are contacted at two places of a human body, it is possible to efficiently move electrons. Therefore, it is possible to effectively eliminate excess or deficiency of ions and electrons in a human body and effectively remove static electricity from the human body. Moreover, a current-backflow prevention unit can cut off a high-frequency current from a radio unit flowing between a human body and each branch line. Therefore, it is possible to prevent the influence of a high-frequency current and stably obtain a high effect for eliminating excess or deficiency of ions and electrons in a human body.

It is preferable that a human body earthing apparatus of the present invention has a current direction controller connected to the other end in series. Moreover, it is preferable that the current direction controller has a first diode for supplying current from one end of the earth line to the other end of the earth line, a second diode for supplying current from the other end of the earth line to one end of it, a switching earth line for supplying current in the both directions, and a switch capable of selectively switching the first diode, second diode, and switching earth line. In this case, when electrons are insufficient due to chronic cloddish sense or paralysis, chronic cloddish sense or paralysis can be reduced or eliminated by switching the switch to the first diode and flowing current from a human body to the ground and moving electrons from the ground to the human body. When electrons are excessive due to pain, the pain can be reduced or eliminated by switching the switch to the second diode, flowing current from the ground to the human body, and moving electrons from the human body to the ground. Moreover, when excess or deficiency state of ions and electrons is unclear or static electricity is electrified, the uncomfortable sense can be reduced or eliminated by switching the switch to a switching earth line so that current can be flown in the both directions and electrons can be moved in the both directions. Thus, by controlling the moving direction of electrons, it is possible to efficiently eliminate excess or deficiency of ions and electrons in a human body and eliminate static electricity from the human body.

In the case of a human body earthing apparatus of the present invention, it is preferable that the high-frequency-current limiter has a configuration capable of selectively switching the frequency band of a high-frequency current which can be cut off. In this case, even if the frequency of the high-frequency current flowing through an earth line is changed depending on a place, it is possible to cut off the high-frequency current correspondingly to the change. Therefore, it is possible to effectively prevent the influence of the high-frequency current and stably obtain a high effect for eliminating excess or deficiency of ions and electrons in a human body.

It is allowed that a human body earthing apparatus of the present invention has a DC power supply and a circuit selection switch, the circuit selection switch is constituted so as to be able to selectively connect each branch line to the DC power supply and the earth line, and one of the branch lines is connected to the negative terminal of the DC power supply and the other of the branch lines is connected to the positive terminal of it through the circuit selection switch.

In the case of the above configuration, when connecting the circuit selection switch to the DC power supply, it is possible to circulate DC between a human body and each branch line. Thereby, it is possible to change a chronic tissue. Thereafter, by switching and earthing the circuit selection switch, it is possible to effectively reduce or eliminate chronic pain.

It is preferable that a human body earthing apparatus of the present invention has a heating portion for heating the human body connecting portion. The heating portion is constituted of a keep-warm plate or a consumable body warmer housing portion so as to contact with and warm the human body connecting portion. When the heating portion is used, it is possible to improve the effect for eliminating excess or deficiency of ions and electrons by warming and perspiring a contact portion such as a foot so that current easily flows by the perspiration.

According to the present invention, it is possible to provide a human body earthing apparatus capable of obtaining a high advantage stably without using exclusive technique or knowledge.

### BRIEF DESCRIPTION OF THE DRAWINGS

FIG. 1 is a circuit diagram showing a human body earthing apparatus of an embodiment of the present invention;

FIG. 2 is a block circuit diagram showing an operating state of the human body earthing apparatus shown in FIG. 1; and

FIG. 3 is a circuit diagram showing a modification having a DC power supply of the human body earthing apparatus shown in FIG. 1.

### DETAILED DESCRIPTION OF THE PREFERRED EMBODIMENT

An embodiment of the present invention is described below by referring to the accompanying drawings.

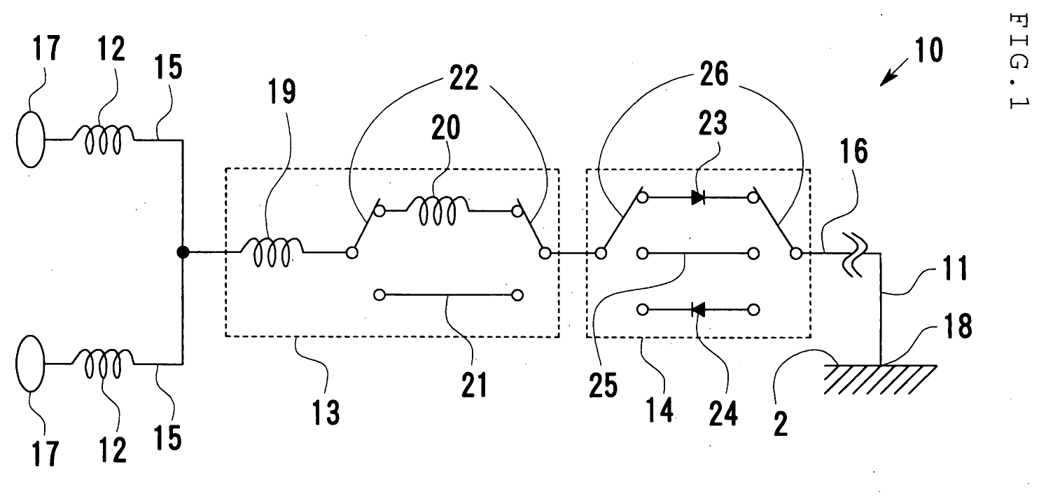

FIGS. 1 to 3 show the human body earthing apparatus of the embodiment of the present invention.

As shown in FIG. 1, a human body earthing apparatus 10 has an earth line 11, two current-backflow-prevention units 12, a high-frequency-current limiter 13, and a current direction controller 14.

The earth line 11 is constituted of an insulated cord and has a length of several to 10 m. One end of the earth line 11 is constituted of two branch lines 15 and the other end of the line 11 is constituted of one main line 16. The earth line 11 has a human body connecting portion 17 at the end of each branch line 15 and an earth connecting portion 18 at the end of the main line 16. Each human body connecting portion 17 is electrically connected to the earth line 11 and constituted of a conductive elliptic metallic plate. It is preferable that each human body connecting portion 17 is particularly constituted of stainless steel. The earth connecting portion 18 is electrically connected to the earth line 11 and constituted of a clip.

Each current-backflow prevention unit 12 is constituted of a line filter using ferrite beads for a coil and connected to each branch line 15 in series.

The high-frequency-current limiter 13 is connected to the main line 16 in series. The high-frequency-current limiter 13 is constituted of two line filters 19 and 20, a switching cord 21, and a two-point interlocking switch 22. One line filter 19 is connected to the main line 16 in series. The two-point interlocking switch 22 is connected to an end of the main line 16 in series so as to be able to selectively switch the other line filter 20 and the switching cord 21. The line filters 19 and 20 are respectively constituted of using ferrite beads for a coil.

The current direction controller 14 is connected between the high-frequency-current limiter 13 and an end of the main line 16 in series. The current direction controller 14 is constituted of a first diode 23, a second diode 24, a switching earth line 25, and a three-point interlocking switch 26. The first diode 23 is connected by turning an anode to one end of the earth line 11 and a cathode to the other end of the line 11 so as to move electrons from the other end of the earth line 11 to one end of the line 11. The second diode 24 is connected by turning a cathode to one end of the earth line 11 and an anode to the other end of the line 11 so as to move electrons to from one end to the other end of the line 11. The switching earth line 25 is constituted of a cord line so that electrons can move in the both directions. The three-point interlocking switch 26 is constituted so as to be able to selectively switch the first diode 23, second diode 24, and switching earth line 25.

Then, functions are described below.

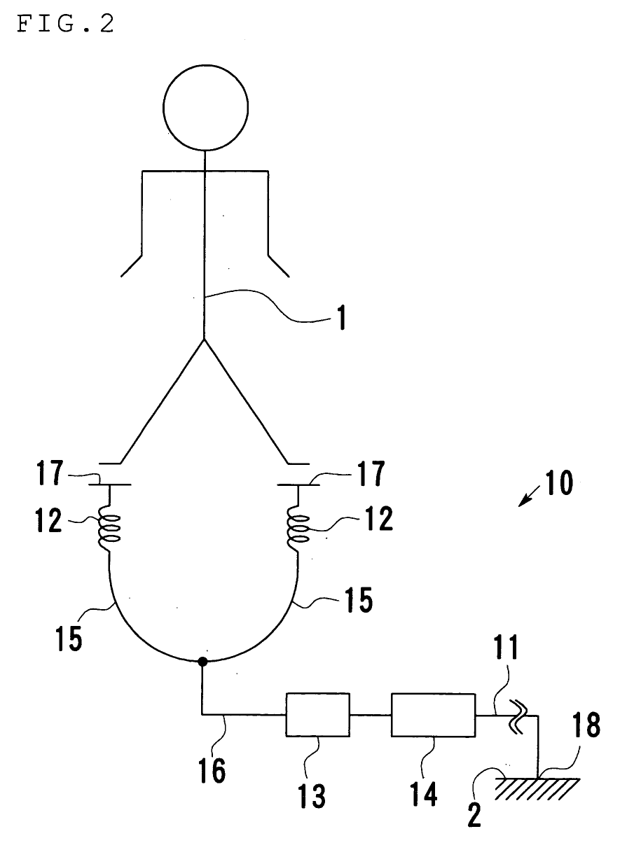

As shown in FIG. 2, the human body earthing apparatus 10 is used by bringing each human body connecting portion 17 in contact with the arch at the back of foot or pain or paralysis portion and connecting the earth connecting portion 18 to an earth terminal in a room. In the case of the human body earthing apparatus 10, when electrons are insufficient due to chronic cloddish sense or paralysis, electrons are moved from ground 2 to a human body 1 by switching the three-point interlocking switch 26 to the first diode 23. When electrons are excessive due to pain, electrons are moved from the human body 1 to the ground 2 by switching the three-point interlocking switch 26 to the second diode 24.

Moreover, when the state of excess or deficiency of ions and electrons is unclear or static electricity is electrified, the three-point interlocking switch 26 is switched to the switching earth line 25 so that electrons can move in the both directions. In this case, electrons are moved from the human body 1 to the ground 2 or from the ground 2 to the human body 1 in accordance with the state of excess or deficiency of ions and electrons in the human body 1. Thus, the human body earthing apparatus 10 can efficiently eliminate excess or deficiency of ions and electrons in the human body 1 or remove static electricity from the human body 1 by controlling the moving direction of electrons. Moreover, because two human body connecting portions 17 are contacted at two places of the human body 1, it is possible to efficiently move electrons. Thereby, it is eliminate pain and paralysis in the human body 1 due to excess or deficiency of ions and electrons.

In the case of the human body earthing apparatus 10, the high-frequency-current limiter 13 can cut off a high-frequency current from a radio unit flowing through the earth line 11. Moreover, it is possible to selectively switch the frequency band of a high-frequency current which can be cut off by switching the two-point interlocking switch 22 to the line filter 20 or switching cord 21. Thereby, even if the frequency of the high-frequency current flowing through the earthline 11 is changed depending on a plate, it is possible to cut off the high-frequency current correspondingly to the change. Particularly, because the earth line 11 has a length of several m to 10 m and a high-frequency current from a radio unit easily flows through the line 11, it is possible to effectively cut off a high-frequency current by the high-frequency-current limiter 13. Therefore, it is possible to effectively prevent the influence of the high-frequency current and stably obtain a high effect for eliminating excess or deficiency of ions and electrons in the human body 1.

In the case of the human body earthing apparatus 10, the current-backflow prevention unit 12 can cut off a high-frequency current from a radio unit circulating between the human body 1 and each branch line 15. Therefore, it is possible to prevent the influence of the high-frequency current and stably obtain a high effect for eliminating excess or deficiency of ions and electrons in the human body 1.

Thus, the human body earthing apparatus 10 can be easily used because exclusive technique or knowledge like the case of acupuncture or moxibustion is unnecessary.

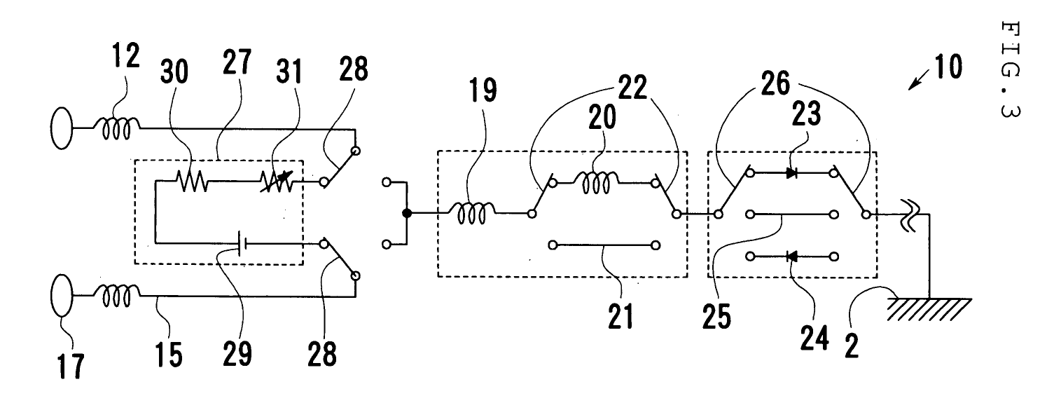

As shown in FIG. 3, it is allowed that the human body earthing apparatus 10 has a DC power supply 27 and a circuit selection switch 28. The DC power supply 27 is constituted of connecting a dry cell 29 a resistance 30 and a variable resistance 31 in series and the circuit selection switch 28 is constituted of a two-point interlocking switch so as to be able to selectively connect each branch line 15 to the DC power supply 27 and the other end of earth line 11. One branch line 15 is connected to the negative terminal of the DC power supply 27 and the other branch line 15 is connected to the positive terminal of the DC power supply 27 through the circuit selection switch 28. In the case of the configuration shown in FIG. 3, by connecting the circuit selection switch 28 to the DC power supply 27, it is possible to circulate DC between the human body 1 and each branch line 15. Thereby, it is possible to change a chronic firm tissue. Thereafter, by switching the circuit selection switch 28 to the other end and earthing it, it is possible to effectively reduce or eliminate a chronic pain.

Medical treatment examples using the human body earthing apparatus 10 are shown below.

1. Case in which acute pain, spontaneous pain, and working pain were clearly complained

A medical treatment was performed in which the three-point interlocking switch 26 was switched to the second diode 24 to move electrons from the human body 1 to the ground 2 because electrons in the human body 1 were excessive.

[Case 1] Tenosynovitis of Right Thumb, 62 Years Old, Female

She had a right thumb tenosynovitis since half year before she visited this hospital. An injection was applied to the local place in orthopedic surgery. Medicine was dosed to her in internal medicine but here symptom was not improved.

A pain runs when she extended the thumb. As a result of warming the thumb for two to three days, she came to this hospital while saying that pain was increased. She had a slight spontaneous pain.

As a result of setting the three-point interlocking switch 26 of the human body earthing apparatus 10 to the second diode 24 and setting the human body connecting portion 17 to her, the pain was immediately halved. The symptom is improved by the medical treatment of five times every other day until she hardly felt the pain and was cured.

[Case 2] Knee Arthritic Pain, 65 Years Old, Male

After he climbed a mountain two weeks before he came to this hospital, he felt a pain of his right knee. Though he came to this hospital and received an injection, his symptom was not improved and it was diagnosed that there was no way because he aged and his cartilage was decreased. There was an extreme pressure pain at the right bottom of his knee joint.

As a result of setting the human body earthing apparatus 10 to him, switching the three-point interlocking switch 26 to the second diode 24, and pressing a portion at which he felt a pain, he hardly felt a pain. Because he said that a pain was left when went down stairs, medical treatment was applied to him at a pace of twice per week. The pain was eliminated at the sixth time and he was cured.

[Case 3] Slipped Disk, 54 Years Old, Male

A pain ran through his waist when he raised a thing before the day before yesterday of coming to this hospital and he came to this hospital while he could not perform backbend but he could only slouch.

He was put on a bed and his waist is drew up by to designate raising this waist. However, his waist could not be raised due to a pain. As a result of setting the human body earthing apparatus 10 to him and switching the three-point interlocking switch 26 to the second diode 24 to designate him to move his waist, his waist was considerably raised though he felt a pain.

[Case 4] Atopic Dermatitis, 25 Years Old, Female

She had a symptom that the itch and tumescent of her lower body (particularly waist portion) got heavy before menses and she could not be motionless.

She was laid on her stomach to cover the waist where the itch was heaviest with an aluminum foil and the three-point interlocking switch 26 of the human body earthing apparatus 10 was switched to the second diode 24 and the human body connecting portion 17 was connected to the aluminum foil. After 30 min, she was improved to a state in which she hardly felt the itch.

[Case 5] Pain After Wrist Born was Fractured, 43 Years Old, Male

He has a pain on the portion of the wrist whose born was fractured in the past. An aluminum plate was bent to closely contact it with the wrist. When switching the three-point interlocking switch 26 of the human body earthing apparatus 10 to the second diode 24 and connecting the human body connecting portion 17 to the aluminum plate, the pain is mostly eliminated even if bending the wrist. Even after removing the aluminum plate, the pain was decreased up to approx. 30% compared to the state before medical treatment and her symptom was improved.

2. Case in which chronic cloddish, paralysis sense, boredom were complained. A medical treatment was performed in which electrons were insufficient in the human body 1 and the three-point interlocking switch 26 was switched to the first diode 23 to move electrons from the ground 2 to the human body 1.

[Case 6] Uncomfortable Feeling After Extraction of Tooth, 35 Years Old, Male

He has had an uncomfortable dull pain after extraction of two teeth at the right back for five years. He took every pain-killer at a hospital. Though an anesthetic injection was applied to his local place, there was no effect.

The human body earthing apparatus 10 is set to him and the three-point interlocking switch 26 was switched. The first diode 23 was set and left as it was for 15 min. Then, the dull pain was decreased to approx. 60% and his symptom was improved.

[Case 7] Boredom and Paralysis After Cold, 74 Years Old, Female

She had a symptom of cold continued for two weeks or more. Though her cough was cured, she had a symptom in which lassitude remained, feet were numbed, and strength of the feet was lost.

The human body earthing apparatus 10 was set to her and the three-point interlocking switch 26 was switched. The first diode 23 was set and left as it was for 15 min. After medical treatment, the feet recovered strength and her symptom was improved to the state in which she did not feel paralysis.

[Case 8] Foot Paralysis While Anticancer Drug Was Used, 56 Years Old, Female

Her breast cancer was transferred to her waist and she took anticancer drug through hospital visit. Her foot bottoms were numbed in addition to alopecia, nail necrosis, and boredom.

The human body earthing apparatus 10 is set to her. The three-point interlocking switch 26 was se to the first diode 23. When 15 min passed, foot paralysis was halved and boredom was decreased and her symptom was improved.

3. Cases of health control and fatigue recovery and case of complaining subacute pain and muscle pain

Medical treatment was performed by switching the three-point interlocking switch 26 to the switching earth line 25 so that electrons can move in both directions.

[Case 9] Dull Pain from Neck to Hand After Whiplash, 53 Years Old, Female

She had a neck sprain due to a traffic accident half year before visiting this hospital. Thereafter, though she visited an orthopedic surgery, her symptom was not improved. As a result of complete check, it was diagnosed that the paralysis could not be eliminated because hernia was recognized on the neck and she visited this hospital.

When making her turn upward while setting the human body earthing apparatus 10 to her and switching the three-point interlocking switch 26, the symptom almost disappeared by the switching earth line 25. When removing the earth line 25, pain and paralysis were halved. The symptom almost disappeared and was cured through six times of the medical treatment same as the above.

[Case 10] Tiredness and Muscle Pain After Playing Golf 78 Years Old, Male

He felt tiredness after playing golf and visited this hospital. During diagnosis, he had a blood pressure of 150/90 and a pulse of 85 and irregular heat beat also appeared.

The human body earthing apparatus 10 was set to him and the three-point interlocking switch 26 was switched to the switching earth line 25 to take a rest for 30 min. He was stabilized at a blood pressure of 130/80 and a pulse of 75. On the following day, the tiredness disappeared and the muscle pain was considerably moderated.

4. Case of asthma attack

The medical treatment was performed in which asthma the three-point interlocking switch 26 was switched to the second diode 24 to move electrons from the human body 1 to the ground 2.

[Case 11] Asthma Attack, Middle to Serious Grade, 55 Years Old, Female

She had the attack since two days before visiting this hospital. In the case of the normal medical treatment, the attack is restrained by continuing two asthma curing drip infusions per day for approx. three days.

As a result of setting the human body earthing apparatus 10 to her and switching the three-point interlocking switch 26 to the second diode, the symptom of the asthma attack was decreased to approx. 30% before medical treatment and moderated. As a result of performing drip infusion to make doubly sure, one drip infusion was enough.

[Case 12] Asthma Attack, Serious Grade, 36 Years Old, Male

He took a cold and caused asthma attack together. He also had a throat pain. As a result of applying three asthma curing drip infusions to him, his symptom was not improved.

As a result of setting the human body earthing apparatus 10 to him and switching the three-point interlocking switch 26 to the second diode, the throat pain was moderated and the rigor of breathing was decreased to 20 to 30%. After he was kept quiet in bed for 30 min, the human body earthing apparatus 10 was removed. Also thereafter, a state was kept in which the rigor of breathing was decreased to 20 to 30%.

5. Medical treatment was performed by using the human body earthing apparatus 10 and the ion pumping cord disclosed in Japanese Utility Model Laid-Open Nos. 50-64387 and 50-64388.

[Case 13] Slipped Disk, 60 Years Old, Female

When she was mowing the grass 2 days before visiting this hospital, she felt a sudden pain at waist and thereafter, she felt a pain whenever performing an operation.

When switching the three-point interlocking switch 26 to the second diode 24 and setting the human body earthing apparatus 10 to her, working pain was halved. She was turned on her stomach and a needle was applied to the portion on which a pain was left and an ion pumping cord was connected from the needle to the human body connecting portion 17. On the following day, the pain was decreased but she still felt a pain at the right side of her body. Therefore, medical treatment was performed by the same treatment technique by changing the ion pumping cord disclosed in Japanese Utility Model Laid-Open Nos. 50-64387 and 50-64388 to the human body earthing apparatus 10. As a result, the pain of the right side of her body was halved and the pain almost disappeared and her symptom was cured through the medical treatment at the third-time hospital visit.

In this case, because the high-frequency-current limiter cut off the high-frequency current from a radio unit flowing through the cord, it was estimated that a high effect was stably obtained.

6. Case of complaining chronic pain. Medical treatment was performed by using the human body earthing apparatus 10 shown in FIG. 3.

[Case 14] Chronic Waist Pain, 50 Years Old, Female

She visited this hospital because she felt heavy at her waist while she was doing the work of care.

As a result of applying medical treatment to her by switching the circuit selection switch 28 of the human body earthing apparatus 10 to the other end, the using the heaviness of the waist was decreased to approx. 30% and thereafter, her symptom was not changed.

The circuit selection switch 28 was switched to the DC power supply 27. After 10 min passed, the circuit selection switch 28 was switched to the other end and the three-point interlocking switch 26 was switched to the switching earth line 25 and they were further left as they were for 10 min. Then, the heaviness was moderated and her symptom was improved compared to the case of the past medical treatment.

[Case 15] Chronic Waist Pain and Isciatica, 55 Years Old, Female

Though she had a hospital visit history of several years and her symptom was moderated, heaviness of her waist was enhanced at week ends.

As a result of switching the circuit selection switch 28 to the DC power supply 27, and then switching the circuit selection switch 28 to the other end and the three-point interlocking switch 26 to the switching earth line 25, the heaviness of her waist was moderated. Thereafter, the heaviness of her waist was further decreased and her symptom was further improved than ever.

7. Case of asthma. Two human-body earthing apparatuses 10 were used. Medical treatment was applied to her by connecting one of them to her foot and the other one of them to her hand. Breathing levels before and after the medical treatment were measured to confirm the effect by a breathing measuring instrument (electronic spirometer).

[Case 16] Asthma, 48 Years Old, Female

Her asthma symptom appeared since the night before visiting this hospital. Cough was not stopped and she could perform 60% of breathing of the normal state.

One human body earthing apparatus 10 was set to her both feet and the three-point interlocking switch 26 was switched to the switching earth line 25 so that electrons could move in both directions. Thereafter, the three-point interlocking switch 26 was switched to the first diode 23 so as to move electrons from the ground 2 to the human body 1. Moreover, medical treatment was performed by setting the other human body earthing apparatus 10 to the SANSHOUKEI at her left hand (one of 12 routes (paths) through which vital energy flows in oriental medicine, which pass through the outside of a front arm and terminates at the end of a third finger) and switching the three-point interlocking switch 26 to the switching earth line 25 so that electrons could move in both directions. In a short time, cough was blown out and breathing was improved to the degree in which breathing could be performed up to 90% of the normal state. In the case of the breathing measuring instrument, a one-second rate (FEV 1%) was improved from 69.83% before medical treatment to 76.63% after medical treatment.

[Case 17] Asthma, 69 Years Old, Female

She had a strong pain at her throat and had a symptom in which breathing was difficult. Though three drip infusions were applied to her, her symptom was not improved.

Medical treatment was performed by setting one human body earthing apparatus 10 to both feet and switching the three-point interlocking switch 26 to the switching earth line 25 so that electrons could move in both directions. In addition, medical treatment was performed by bringing the other human body earthing apparatus 10 into direct contact with the portion of her throat where she felt a pain and switching the three-point interlocking switch 26 to the switching earth line 25 so that electrons could move in the both directions. After 15 min passed, she was relieved. After 30 min passed, the pain of her throat and heaviness of her breathing were decreased to half or more and her symptom was improved. In the case of the breathing measuring instrument, the one-second rate was improved from 70.13% before medical treatment to 80.41% after medical treatment.

[Case 18] Asthma, 9 Years Old, Male

He visited this hospital for three days because of asthma attack. He received drip infusion and his symptom was considerably improved. However, breathing was still slightly difficult.

One human body earthing apparatus 10 was set to the both feet and the three-point interlocking switch 26 was switched to the second diode 24 so that electrons could move from the human body 1 to the ground 2. Moreover, medical treatment was performed by setting the other human body earthing apparatus 10 to the SANSHOUKEI of his left hand and switching the three-point interlocking switch 26 to the switching earth line 25 so that electrons could move in both directions. His breathing became smooth and his symptom was improved. In the case of the breathing measuring instrument, the one-second rate (FEV 1%) was improved from 65.80% before medical treatment to 76.77% after medical treatment.

According to these cases, the human body earthing apparatus 10 can be expected for the following symptoms.

1. Among pains caused by various causes, a pain caused by excess or deficiency of cations and anions and electrons in a body (local place).

2. An effect can be expected for most acute and subacute pains. Particularly, an effect can be expected for the pain of a terminal cancer.

3. It is known that a blood pressure is raised due to excessive salinity and potassium is effective to lower the blood pressure. Thereby, because it is estimated that the imbalance between potassium and sodium ions causes blood pressure rise, an effect can be expected for a high blood pressure.

4. The diode therapy is effective for atopic dermatitis and asthma attack among allergic diseases. Therefore, it is estimated that an ion balance is related and an effect can be expected.

5. Because a free radical of atoms is related to active oxygen which causes various diseases, an effect can be expected by adjusting excess or deficiency of electrons.

6. An effect can be also expected for boredom. A person feels very comfortable by using the human body earthing apparatus 10. Therefore, an effect for mental relaxation can be expected. Therefore, the human body earthing apparatus 10 can be used for health control not only in the field of medical care but also in homes only by performing preferable earthing.

7. Even if acupuncture or moxibustion is applied to a person while mounting the human body earthing apparatus 10, there is no problem in applying drip infusion to the person in a hospital. When the symptom of the person is halved while mounting the human body earthing apparatus 10, the acupuncture, moxibustion, or drip infusion for eliminating remaining symptom is minimized. Therefore, it is possible to decrease the pain of a patient due to acupuncture or moxibustion, side effect due to dosage of morphine or steroid and economic burden.

8. There is a possibility of widely using the human body earthing apparatus 10 in acupuncture and moxibustion clinics, orthopedic clinics, hospitals, and homes. Moreover, the human body earthing apparatus 10 can be used semipermanently without paying electricity expense.

9. When a pain is increased or inflammation is caused, an effect can be more easily obtained by earthing a right hand for a left foot or a left hand for a right foot than earthing from the foot bottom or the local place of a pain.

10. An effect can be expected for a chronic cloddish feeling and a remaining pain.

Moreover, application of the human body earthing apparatus 10 can be expected for the following symptoms.

1. A lot of steroid is used to control allergic diseases such as atopy and asthma. There is a possibility for making it possible to control the quantity of steroid by using the human body earthing apparatus 10.

2. When it is possible to moderate a pain by using the human body earthing apparatus 10 after a surgical operation in the field of the surgical operation, there is a possibility of decreasing the quantity of a pain killer and quickening the recovery.

3. If the lactic acid value of an athlete relates to an ion balance, there is a possibility of quickening the recovery after exercise.

4. By always using the human body earthing apparatus 10 in a general home, there is a possibility of controlling electrons in a body and sufficiently being useful for prevention of diseases.

1. A human body earthing apparatus comprising:
an earth line having a human body connecting portion at one end and an earthing connecting portion at the other end; and
a high-frequency-current limiter connected to the earth line in series.

2. The human body earthing apparatus according to claim 1, wherein
one end of the earth line is constituted of two branch lines and has a human body connecting portion at the end of each branch line and a current backflow prevention unit connected to each branch line in series.

3. The human body earthing apparatus according to claim 1, comprising
a current direction controller connected to the other end in series.

4. The human body earthing apparatus according to claim 2, comprising
a current direction controller connected to the other end in series.

5. The human body earthing apparatus according to claim 3, wherein
the current direction controller has a first diode for supplying current to from one end of the earth line to the other end of it, a second diode for supplying current from the other end of the earth line to one end of it, a switching earth line for supplying current in both directions, and a switch capable of selectively switching the first diode, the second diode, and the switching earth line.

6. The human body earthing apparatus according to claim 4, wherein
the current direction controller has a first diode for supplying current to from one end of the earth line to the other end of it, a second diode for supplying current from the other end of the earth line to one end of it, a switching earth line for supplying current in both directions, and a switch capable of selectively switching the first diode, the second diode, and the switching earth line.

7. The human body earthing apparatus according to claim 1, wherein
the high-frequency-current limiter has a configuration capable of selectively switching the frequency band of a high frequency current which can be cut off.

8. The human body earthing apparatus according to claim 2, wherein
the high-frequency-current limiter has a configuration capable of selectively switching the frequency band of a high frequency current which can be cut off.

9. The human body earthing apparatus according to claim 3, wherein
the high-frequency-current limiter has a configuration capable of selectively switching the frequency band of a high frequency current which can be cut off.

10. The human body earthing apparatus according to claim 4, wherein
the high-frequency-current limiter has a configuration capable of selectively switching the frequency band of a high frequency current which can be cut off.

11. The human body earthing apparatus according to claim 5, wherein
the high-frequency-current limiter has a configuration capable of selectively switching the frequency band of a high frequency current which can be cut off.

12. The human body earthing apparatus according to claim 6, wherein
the high-frequency-current limiter has a configuration capable of selectively switching the frequency band of a high frequency current which can be cut off.

13. The human body earthing apparatus according to claim 2, wherein
a DC power supply and a circuit selection switch are included, the circuit selection switch is constituted so as to be able to selectively connect each branch line to the DC power supply and the earth line, and one branch line is connected to the negative terminal of the DC power supply and the other branch line is connected to the positive terminal of the DC power supply through the circuit selection switch.

14. The human body earthing apparatus according to claim 4, wherein
a DC power supply and a circuit selection switch are included, the circuit selection switch is constituted so as to be able to selectively connect each branch line to the DC power supply and the earth line, and one branch line is connected to the negative terminal of the DC power supply and the other branch line is connected to the positive terminal of the DC power supply through the circuit selection switch.

15. The human body earthing apparatus according to claim 6, wherein
a DC power supply and a circuit selection switch are included, the circuit selection switch is constituted so as to be able to selectively connect each branch line to the DC power supply and the earth line, and one branch line is connected to the negative terminal of the DC power supply and the other branch line is connected to the positive terminal of the DC power supply through the circuit selection switch.

16. The human body earthing apparatus according to claim 8, wherein
a DC power supply and a circuit selection switch are included, the circuit selection switch is constituted so as to be able to selectively connect each branch line to the DC power supply and the earth line, and one branch line is connected to the negative terminal of the DC power supply and the other branch line is connected to the positive terminal of the DC power supply through the circuit selection switch.

17. The human body earthing apparatus according to claim 10, wherein
a DC power supply and a circuit selection switch are included, the circuit selection switch is constituted so as to be able to selectively connect each, branch line to the DC power supply and the earth line, and one branch line is connected to the negative terminal of the DC power supply and the other branch line is connected to the positive terminal of the DC power supply through the circuit selection switch.

18. The human body earthing apparatus according to claim 12, wherein
a DC power supply and a circuit selection switch are included, the circuit selection switch is constituted so as to be able to selectively connect each branch line to the DC power supply and the earth line, and one branch line is connected to the negative terminal of the DC power supply and the other branch line is connected to the positive terminal of the DC power supply through the circuit selection switch.
